SEQUENCE LISTING (7 sequences)

SEQ ID NO: 1 - DNA, 16 bp, ARTIFICIAL
  ENCRYPTED MESSAGE WHEREIN DNA BASES  REPRESENT CHARACTERS OF ASCII CHARACTER SET
tatgtttctattttac

SEQ ID NO: 2 - DNA, 28 bp, ARTIFICIAL
  ENCRYPTED MESSAGE WHEREIN DNA BASES  REPRESENT CHARACTERS OF ASCII CHARACTER SET
ttagtacatagctatgtacctaactaca

SEQ ID NO: 3 - DNA, 44 bp, ARTIFICIAL
  ENCRYPTED MESSAGE WHEREIN DNA BASES  REPRESENT CHARACTERS OF ASCII CHARACTER SET
ttagtaccttactagctataagctttcctacataggtatgtaca

SEQ ID NO: 4 - DNA, 20 bp, ARTIFICIAL
  ENCRYPTED MESSAGE WHEREIN DNA BASES  REPRESENT CHARACTERS OF ASCII CHARACTER SET
tatttatctatatatttagg

SEQ ID NO: 5 - DNA, 16 bp, ARTIFICIAL
  ENCRYPTED MESSAGE WHEREIN DNA BASES  REPRESENT CHARACTERS OF ASCII CHARACTER SET
tatgtttctattttac

SEQ ID NO: 6 - DNA, 16 bp, ARTIFICIAL
  ENCRYPTED MESSAGE WHEREIN DNA BASES  REPRESENT CHARACTERS OF ASCII CHARACTER SET
tatgtttctatttacc

SEQ ID NO: 7 - DNA, 7924 bp, ARTIFICIAL
  ENCRYPTED MESSAGE WHEREIN DNA BASES  REPRESENT CHARACTERS OF ASCII CHARACTER SET
taaatatttagaaaacaatctcgtggcgatcgcgccatcggctaacctatcgatcgctggtcgcgtatcaacaatcgtcggtcggtcgcgccctacgggctcttcgaaccccgtaggcgacacggcgcggcggatgattgtcgccttgctacccgtggtgcgcccagaccttcgacgctcctggtacctgcgcctcatcgttatctttgttggagtgcaagatggagagtttcccggacgggtagcaagcctgcgtaatatctccaaatgtccaaagcttattgttttcaataacgtgatcctttacctgcacattagtattatcaccagcgtgcacccatgcgggcgccaaccttgctggacttcgacgccgctgtcgttgccctctgagtgaatgattgtgcccactgtggtggggcgcctagtcggtcggtcgaggtgttcattaatggatcgatcgacctatcgaggaatcgatcgatcgatcgggcgatcgcgccatcgatcgatcagtcgtcctacgccggctctctctgcatttcagctcgcttatcgagaggcctgtgcaaggagccctgttacattgggctatctaagacatggggacagtcggccgacagagtataataggaaccacgcctaatggataacagctttcgaaacccactccagagcctgtttactctaattggctccggggctgatggtgagggctgtgaacccggactcccagcctagggagtacagaccatgatccctatgccggattagccctaggctgtcacactaagctatcctcagcgtgagcgtgtccggacttcgcaggctgtgcgtcttgagtgcgcgagtggacgggcgtgcggatccgcgcacgaacgcttcgtcgttcggtcgtcttcacgaccgcccaactttccagccatccaggtagccacgcaagcacatacacatacagacattttataatccactctattatccaatctttctgctgatctgtctacctcgtaggctccctggcttaagtgctaactcaccaaagtcccgacctaccaaccctccgtcttaccaccctcctcgccgcccggctgccctgcccgctatgcgggcagcattgctagccacacagcaagcatcagggcctgcgtcaacgcacgctccgtcggccgggccgctggtcggtgcggaggggggagcgagggtaggcatgtggggtggatcgcgcttggactcctcggctgatttgctgaccgagccgtagaatgatgctcagaaggagatcgagatagacacgatacttatcagtctgtgtgtatgtacgttcgtccgtgcgtgggtaggttggtcgatcgattgatctacgttaatcccactctgcggcgtgacataatgaattacccgccgcccactgtgctgcgaaacccagtttactcagttaatccgactatgccacggtacaaaatatccggggtgcatccgactttgcaaatgaatctaaagcgctacgttattgtaaagatcgtaattaacgaagcggtcgttaattaatctgaggtgcagatgaatacatttaaaccatgcagttattcatcagtcgcatcgcaaacttgtagacgctgaatattaggtatgattaatgatacgcgtgatgacaattacgtgtttaagcgcaattaattctggtagcgttatgcctgtcaaggcggtcctacaactaggttcgatccttacgactggaagatggctctacacacggaccccccaaaccaattatagttacctagtccttaaaaaccatactagtttggctttattgatactaagactaagcttacgtcctgactcgcgattaatggacacacgtttcctgacaagctcctcgggggccatatatatgcctgacgccagaaactggtctcattctcgatatgaagcgacccaaagcgcggtgtatcgttgtcgaatccaactaagatgcatcgcgcgcggcggatcaatcttacgagactcaggtactagtggtatcgtggctgccttgtgacgcttaaatcgtacttcgtcgcgattgattgtattataaacaatcagcaaattaaatcgatggcggactttataaagctaaactacgcctttaagttacgcgctgtgagcagctgaggccggttccttaagttccatacattctatcaatagcgcttcctgcctaggtatgggctctagggctatcttgctaaagttgactcagagagaattacctcggaataaaacaacacgcggcagtcagattttgtcactatttttacgtaactagggtgatctccggaatgtcaactccgggcccccacacgatggtggagatctcctcgcccgtgggcttctggactagacgttagggcatgcacatacgttgacgaaattgttacgcggagacgatagaatttataacctttccaccatctagtatgagggattcatacgctgcccttctcctaataggaacgtacactaaattaattgccgtgctaccaatgcgactactttgggataacggcctgcggttgtcgtcgggtgaactatcctatcgttcgactctatagcaaggcttatcgtgctaactaatttacatagtaggactatcgccacacgggatgcacatacccgactatcgggtcccagagactacgttgaggaaagccaggcttagttttacacattaaccgatggcgtgacggggactttgtcgtcggtacataatcgtcaggtcatcaattcctgctgatatggcgaaattgctgagtatctctatggactaacaactgctaggtgctctggagccgaccgccgcgacatacaagatagacacgtctaaacagctcgttttcatcaacaccatcgtgcatgccgatcgacgtggcacaaacaaattgaatagaaggcatactatatcgtctacttggtatggggcaccttgccgtccaaaaccgttcgaaaaaagatctgtttctaattcatcgtcagtcgatttgaaattctctccccatacgcatggacgcaataagtatcgattggacacctcctcccaggttcaatgtgaagtgacatcgcaacatgaaccccgcggggacagaatgcagtcttccctgcttaatctcgttgggtacagctgaaatgcagtcaggcgcggatgggggcccctcacgggatatggtgataatgtttactagctttacacgtttctagcagaattgcgaaatgacgatagccttccacgcatatgtccttgcctctcacatccgaattggcgatggatgtctctaaatgaattcttatggtcgcgactttaacgcttccaagataacaacagatggtgctcctgaatcacatctcctttgatcttgacatggttccaccctgttccccgggccaacccgttaagccttactatgtgattcgacctaatatggatagtccatccggccatccgtgtacaataatccacagactctgtaatttagaattacatgcactcctctcatcgtatcggcctaatgctaggatcgggtgcgcgattatacggcaactctgtcgatggcctaggttgaagggggatcaacacggtgtacataggccctacagctgacgttcacgtatgatgaatgcttcctcaatgtaatgctcgaatcgagaattctcagtcttaagggcagccatcggagcacgtggcgcggcaatattgattatgacagagctatacagcccactcgggcgatagactgctgagacgcaaacgtgatattaattacgatggctagcattcgacatatcataatcagatattgggtttaggacctttatcgcagtattagtacgatttggtgctgtgcgaaatcttatgtgcgcgtgcgaaacaatatattgttcgaagtgatatgggataggtcagtgtcatataatgtaaatcggttcgtctgacgcgatttaaggctcacattgttatcgctaatcgggatgaacggctcaagtgcagcatggcaccaagattccgagggcaaacgccgcacagtgaggtttggctctcccctctaatatcttacacgtttgtgggattatagggatcacatggccacggcctgtaatattgtcatgtagcccggatgataccggaatactaaaattggaggggttctaggtcatgctaactgctcggggctcatggagttgtagagttatcaacaggatctcggaattcccgtaagcgggatctccttgccgataagtttgtgctgctgcccgtcttcgcgccggaacgcgcttccaaattctccctactaacgcatgctgatgcaccattggagcattctgggatgggcgtttatcgaaacgagtgtttgtctataatgcatgacgaggtctctgctgggtagaattggtgatttggaagcgatacgggttatagtctcacgtactgatggactagtatgcgtgaaggaatcgaatacttcgacacgatgacgtagggagccacgcgatcaaggactgcccagtggtctactatctatcttcaacagattgagggggagcggtgccgctgatttaattttagcatcggtcgctggttaacttttagtatcgcgcctttaaagaatctaatctccgttagtgtcgggttgattttctgcgaaatagaactaattcaattgcttatctgcttgatcgattcggaagccagggtgggtagggtagttacgtacgcctgaatctgaaccatcagtcgtaatgaattactgaagacgcgcgatgcctggataaaattatcgcctatgtcccaactaatggcacgacaggctcagagcatgctactgtgtagtgagatccgcttatcgccccattcgtggtcgcgttatgccactgagtaacaagtgatgtccagtgtctaatacgaccgctcgggtcgatggtcaagcggcacagtgacattaacttttgctttcacattgaacaaattctcccacttcagcacatgtaccccctgctgcatacagaccaggtcttttgtccacaccttgcacgggtgcctgaatgcctttccgctggcctaagccagtgacgtgaatgtaaagagcgctcgcactgtagtcatggagaattataatcgatagataaatacgtggcgcaccaccccaacatcctcgcgggctgttactagaaattgtgtataccgtgggggtgattaaaaaatggtgagacgtgctgtatggtctttgtgatctctgctactattgggtgctgcataaatcgtacctccaacttgaggcatcatagctacggaacccgtaaaattggtcatatacgcaaacacaacagtaagtaggtggagccgaagtgctctcgtggccgaagacaacaacctttgcccatgccttaaagactgcgtgataaccgtcttcccatcaggaggtgaaggcgatatggtaatctataggtattgatggcaagaggtcggaacccagcttactcgatagcgttgtcgatcgcgcttcctgtgctccttcctacaaagtgggatagcatcatagacaggcatccgggtccaatcgccgaacgcgtcacgcatcgcatgattaattacagtgtcgcattacatctagtatgtattaggtgggcaccgcggtacagcatggacaggcgctcacggacacaaaaacgcgtcaacaaaagttaggtatgggtggcgccaggtgaaaacgccagctctgctatggtcctaagtaattgcagcatgtcttgagatctcatagctaccgtcttcagaacgatattagctaactttcccttccgtctcattacttatgcgggcttcatcgcggttaccggctggtaagatacgtaagctacactagtaagcatactgcaggtatgagccgatcctgcaattacccatattggtttttgtatttacacgtatggcgattacacttcttaaactagaactcgtttactaattcttcgttcatactcatggcaatagcatgatctcgtattaccatgttatacgtagtcatagtgtgccaacagtacgttaacctacaatgctccacgccgaccttgtagaacagcatgatactatatacccgggcatcgcgcaccgataactgcagatcatggaatgaccgctctacgtggatttaactcgggtggccctatagataaatattcttaccaccgccctgggatatataggccgtcagcacgtttatgtcctagtacgcagtacgcgcctattaatataacagctgtcagtaagggtccagaattctagggccgatgaattacaagcaggtgaatagatacgattgggatattatcacaacaactcgcgaatggattatcagtacgagccacggcccagcacattattcaccaacgggattaggtgacgccagtgcgtgctgctactacaatgcatcgcgggtgttgacggttaaggtagctcgggcgcgatagatgatactggcccgagaccagtttctctatattaacctagtaagacaggcctggcccggaaaccgtttctgtaccccgacctagtataagactactgggccgctagcggactattgacaaatcgcgcgtagaaaatgcctgggccgtctgccgtcggtttctttagctataccttgtaattaaatactggaccaaccacagtttcttcagagtaaccttgtactttaggcctttacatcgtcctccttctccaacacgaccttgtagctcactactggtccacaggcagtttcttcagcaccagcttgtatctgatgcctggtccattgtccccttctccaatcgtagcttgttcccgaatactggtgctatgcctaattctagtagataacctcgttaccaagctcgtttgcttcaaaagtctcttgttcccgacgacgtagccaatagcgggcgctcgttcagtctctcgagctctccagcgttggccatgcctttcgctagtccgccctctggtcctatacctggttcccccgagcgggggccaacacacacgctgctctcaaagctggttcaggagcgctggacccttccaagtctctaatgcagtctctagttgagatttactggagccatgctcccctcttatgacaactgaggttatgttagcctggagcttagataccctctcacgcgccctgacgttctattgtagtggaactacattcccgtcccacgataactgacgtcgtactcgcgtggaacactagtaccgtccgacaccggcggatgtcttagtttagtggtacttgtcgcccttccaacaaaagaagacgtctcaatagcgtggtaccgtttttccgtcctactctcacggagatcactatgtagtttcagcgtcagggtgtcctttaaaacatagaatccgttaggaggtttaggggccccccgtccctctcacgacgaaataataaataggggggagctcggacccgtccgtcataccagagaatctaagggctgggggaggattagaccgtccatcctgtcaaaggatgcacgtgcagaggaagagtacacccatcccagcgaaaagtctatcctcatcctgggggtcctgaaaaccatcctctgtctgagagtatgttgaggagcgggatgatggcgaccctccccaaccggggccctctggtccgcctatagtttcagagatgaattagctaaggttgtagcttattttccatagggttttgctccggaccatccggtcgtgtagcgcgattgacttgccgggttgtgtccccgtatccaggtcacgacctcatggggaactagtggctgtccggcagtatcctggtacgcacctcatgtggtatgcgtggctgttggtccgtatatggacctatatatggatcgaagc

(19) United States
(12) Patent Application Publication
(10) Pub. No.: US 2005/0053968 A1
(43) Pub. Date: Mar. 10, 2005

(54) Method for storing information in DNA

(75) Inventors: Lalit M. Bharadwaj, Chandigarh (IN); Awdhesh Kumar Shukla, Chandigarh (IN); Amol P. Bhondekar, Chandigarh (IN); Rakesh Kumar, Chandigarh (IN); Ram Prakash Bajpai, Chandigarh (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 10/812,839

(22) Filed: Mar. 30, 2004

Related U.S. Application Data
(60) Provisional application No. 60/459,140, filed on Mar. 31, 2003.

(51) Int. Cl. C12Q 1/68; G06F 19/00; G01N 33/48; G01N 33/50

(52) U.S. Cl. 435/6; 702/20

(57) ABSTRACT

DNA is a natural molecular level storage device. Molecular storage devices use each molecule or part of it for storing a character. Thus it is possible to store information million of times than presently used storage devices. For example a JPEG image (i.e. flag of India) having file size of 1981 Bytes can be encrypted using 7924 DNA bases which occupies about 2694.16 nanometers In other words flag of India can be encrypted 8.07×10⁵ times in human genome which comprises 6.4×10⁹ DNA bases and occupy a tiny volume of about 0.02 μm³. A method for storing information in DNA has been developed which includes software and a set of schemes to encrypt, store and decrypt information in terms of DNA bases. The main advantages of the present method over exiting art is that it addresses complete set of extended ASCII characters set and thereby, encryption of all kind of digital information (text, image, audio etc.). First of all, information is, encrypted along with carefully designed sequences known as header and tail primers at both the ends of actual encrypted information. This encrypted sequence is then synthesized and mixed up with the enormous complex denatured DNA strands of genomic DNA of human or other organism.

### FIELD OF THE INVENTION

The present invention relates to a method for storing information in DNA The method of invention comprises storing information in DNA. The present invention addresses storage for all kind of digital information whether it is a text file, an image file or an audio file. Large sequences are divided into multiple segments.

### BACKGROUND OF THE INVENTION

DNA is the best molecular electronic device ever produced on the earth because DNA can store, process and provide information for growth and maintenance of living system. AU living species are as a result of single cell produced during reproduction. In most of the cases this single cell does not have most of the materials required for fabricating a living system but contains all the information and processing capability to fabricate living spaces by taking materials from environment, for example, fabrication of baby from Zygote which contains rearranged DNA sequences of parents. DNA is ready to use nanowire of 2 nm and can be synthesized in any sequence of four bases i.e. ATGC. DNA of every living organism (micro/macro) consist of large number of DNA segments where each segment represents a processor to execute a particular biological process for growth and maintaining life. Other important characteristics of DNA which makes it material of choice for future molecular devices are: DNA the building block of life, can store information for billion of years. The tremendous information storage capacity of DNA can be imagined from the fact that 1 gram of DNA contains as much information as 1 trillion CD's¹ four bases (A,T,G,C) instead of 0 and 1, extremely energy efficient (10¹⁹ operations per joule), synthesis of any imaginable sequence is possible and semiconductor are approaching limit.

Clelland et al, 1999[2], and Bancroft, et al. 2001[3] [U.S. Pat. No. 6,312,911], have developed the DNA based steganographic technique for sending the secret messages. Although their prime objective was steganography (the art of information hiding), they used. DNA as storage an transmission device for secret message. They encrypted the plaintext message into the DNA sequences and retrieved the message using the encryption/decryption key. They used three DNA bases for representing a single alphanumeric character, as DNA has 4 bases (A, T, C, G) so a maximum of 64 (4×4×4) ASCII character can be formed using this scheme. Whereas, a total of 256 extended ASCII characters are required to represent complete set of digital information. Hence, Clelland's scheme cannot be used to address complete set of digital information and has limited scope.

### OBJECTS OF THE INVENTION

The main object of the present invention is to develop a comprehensive DNA based information storage technique.

Another object of the present invention is to encrypt complete extended ASCII character set in terms of minimum number of DNA bases.

Another object of the present invention is to develop software to encrypt/decrypt data in terms DNA bases.

Yet another object of the present invention is to design suitable primers to be flanked at both ends of the encrypted and synthesized information.

### SUMMARY OF THE INVENTION

The present invention provides a method for storing information in DNA The method of invention comprises storing information in DNA. The present invention addresses storage for all kind of digital information whether it is a text file, an image file or an audio file. Large sequences are divided into multiple segments

### BRIEF DESCRIPTION OF THE ACCOMPANYING DRAWINGS

FIG. 1a, Information storage in DNA. Structure of prototypical single segment information storage in DNA strand.

FIG. 1b. Information storage in DNA. Structure of prototypical multi segment information storage in DNA strand.

FIG. 2. Encryption of extended ASCII character set in terms of DNA bases

FIG. 3. Encryption Key. Extended ASCII characters in terms of DNA strands

FIG. 4. Process sheet for encryption & storage

FIG. 5. Process summary

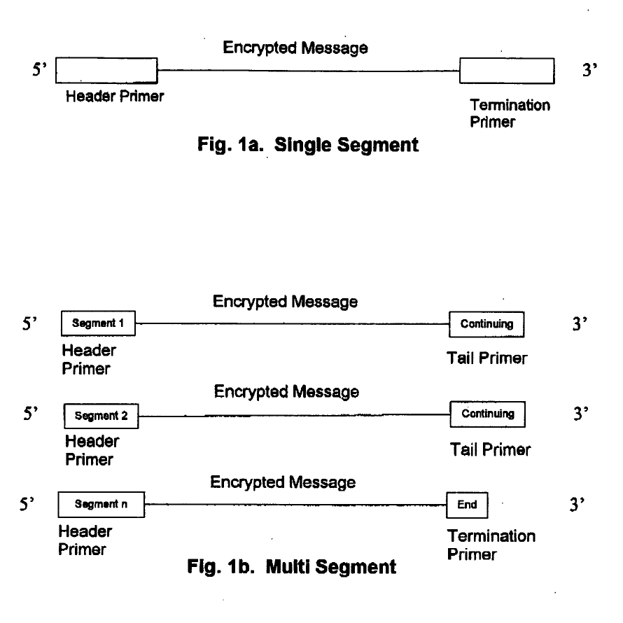

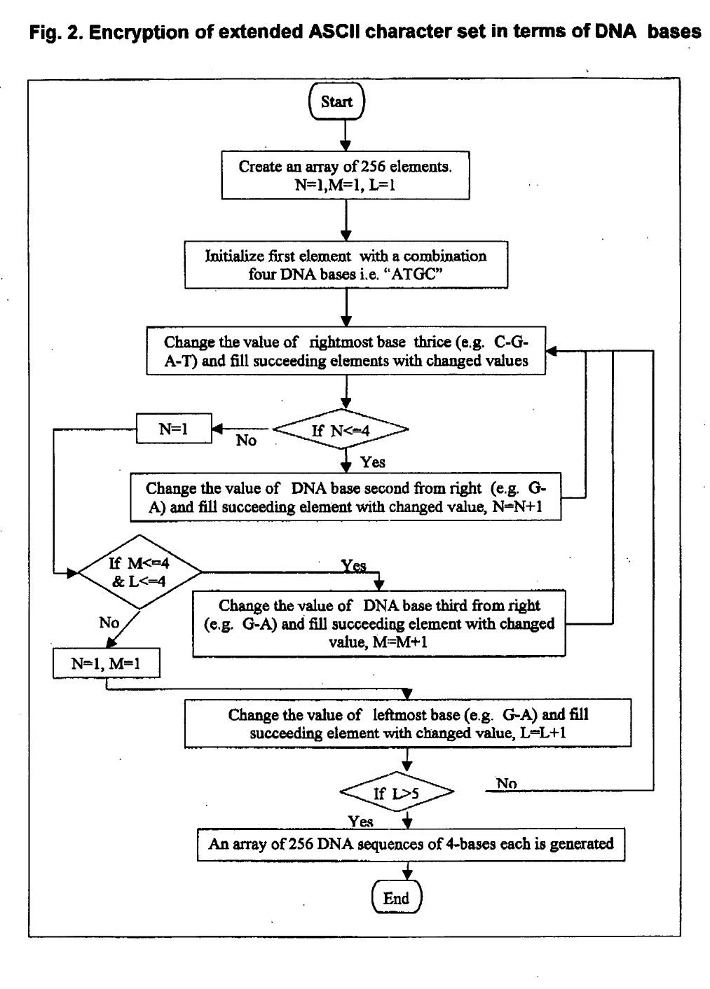

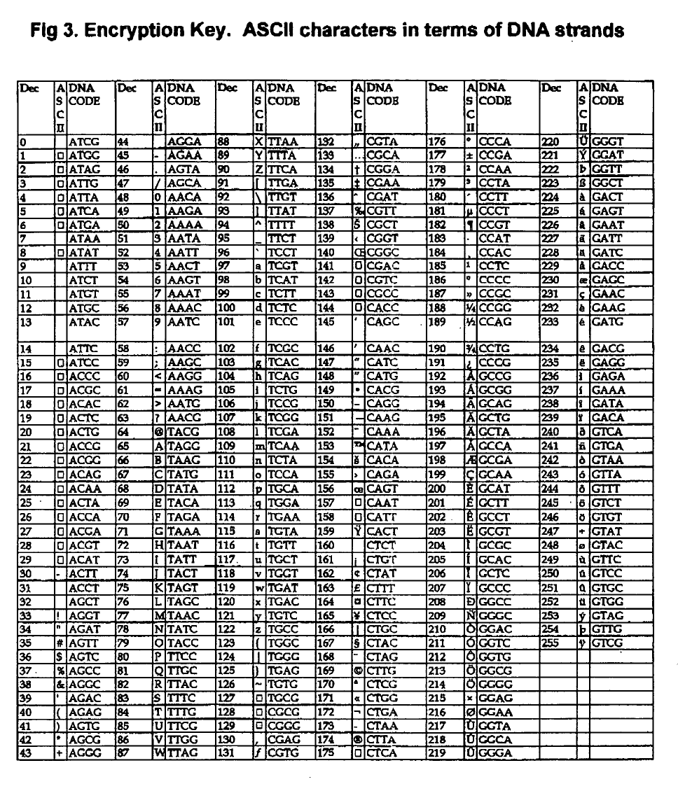

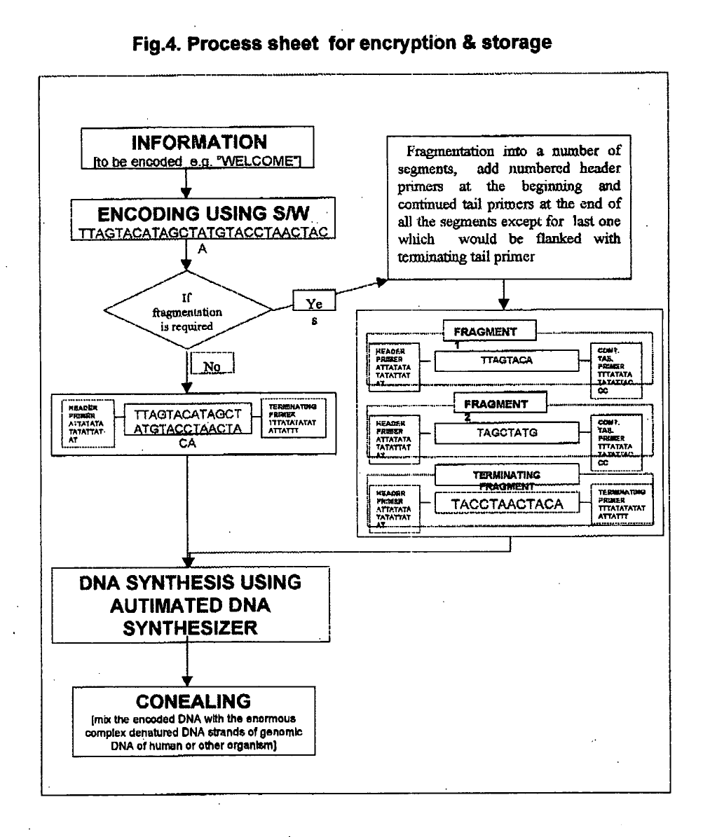

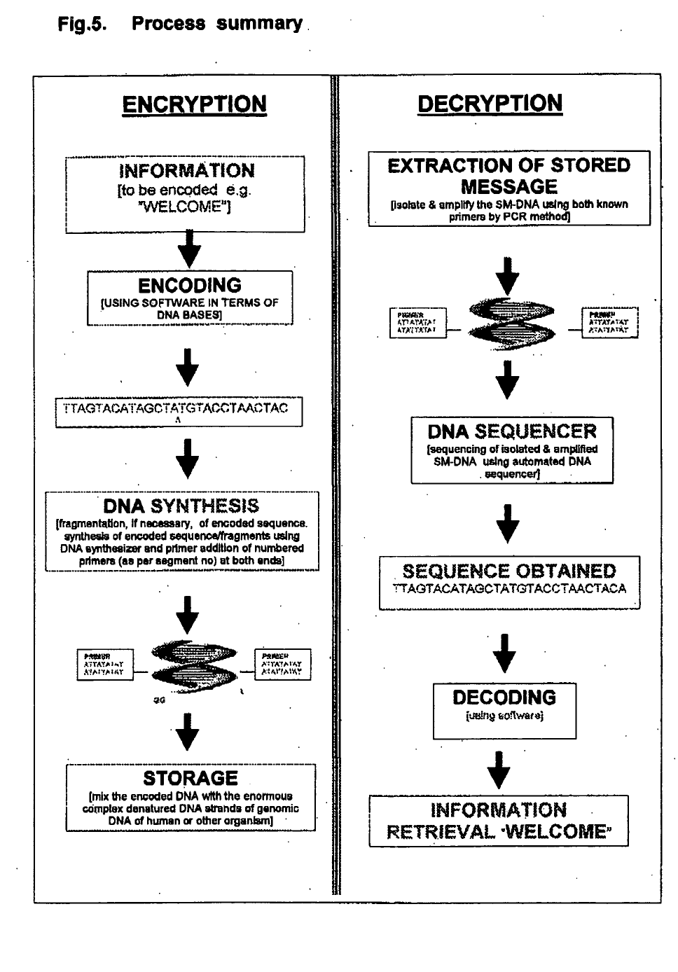

### DETAILED DESCRIPTION OF THE INVENTION

The present invention provides a method for storing information in DNA. The method of invention comprises storing information in DNA. The present invention addresses storage for all kind of digital information whether it is a text file, an image file or an audio file. Large sequences are divided into multiple segments.

The method enables the storage of information in DNA. In another embodiment a software based on the above method enables all 256 Extended ASCII characters to be defined in terms of DNA sequences. The basic concept used is to take minimum number of bases to define each Extended ASCII character. With simple permutation we have 4 sequences combinations with one base Le. A, T, G, C. Similarly, with 2 bases we have 4×4=16 different sequences, with three bases we get 4×4×4=64 distinct sequences and flour bases give 4×4×4×4=56 distinct sequences. Therefore, with a set of 4 bases, complete extended ASCII set has been encoded. Software named as “DNASTORE” has been developed in Visual Basic 6.0 for encryption and decryption of digital information in terms of DNA bases. Using DNASTORE complete extended ASCII character set can be encoded 256 different ways.

In yet another embodiment in our scheme, plain text/image or any digital information is encrypted in terms of DNA sequences using encryption key (software). If the information overflows the limits i.e. it cannot be synthesized in a single piece then it is encrypted and fragmented in a number of segments. Synthesis of encrypted sequence(s) is carried out using DNA synthesizer.

In yet another embodiment a fixed number of different DNA primers sequence have been designed and assigned a number, which resembles the segment position it represents e.g. segment 1, segment 2 . . . segment n. These are called as header primers. Two tail primers have also been designed one resembles continuation and other resembles termination segment.

In yet another embodiment the DNA segment(s) is/are flanked by known PCR primers [as described earlier] at both the ends i.e. header primers are attached at the beginning of segment and tail primers are attached at the end of the segment. If there is only one segment, at the beginning it is, flanked by header primer number 1 and at the end it is flanked by termination tail primer. However, if there are more than one segments, each segment would be attached with header primers numbered as 1, 2, 3 . . . n respectively, at the end these would be attached with a continuation tail primer except for last segment which would be attached with a termination tail primer.

The SM DNA is then mixed with the enormous complex denatured DNA strands of genomic DNA of human or other organism. As the human genome contains about 3×10⁹ nucleotide pairs, fragmented & denatured human DNA provides a very complex background for storing the encrypted DNA. The DNA can be stored and transported on paper, cloths, buttons etc.

In still another embodiment only a recipient knowing the sequences of both the primers [starting and tail] would be able to extract the message, using PCR to isolate & amplify the encrypted DNA strand. Isolated and amplified DNA can then be sequenced using automated DNA sequencer. The DNA sequence obtained can then be converted into digital message using encryption/decryption key (software key).

In yet another embodiment the key is helpful in the secret & secure transfer of information particularly for spying and military purposes. It may also be helpful in anti-theft, anti-counterfeiting product authentication, copyright infringements etc.

TABLE 1
Comparison of present art with existing art

| S. | Existing art | |
|---|---|---|
| No. | Clelland et al., Bancroft, et al. | Reported invention |
| 1. | Uses unique 3-base sequence for | Uses unique 4-base sequence |
| | each alphanumeric character | for each alphanumeric character |
| 2. | Can represent a maximum of 64 | Can represent a maximum of |
| | (4 × 4 × 4) characters | 256 (4 × 4 × 4 × 4) characters |
| 3. | Can represent only ¼^{th} of | Can represent complete extended |
| | extended ASCII character set | ASCII character set |
| 4. | Cannot be used encrypt | Can be used encrypt complete |
| | complete digital information | digital information as shown |
| | i.e. meant for alphanumeric | in examples |
| | characters only | |

### EXAMPLE 1

Encryption and decryption of a textual message “CSHU” in terms of DNA bases may be defined as
a) Generation of an array of 256 elements (unique abase per character i.e. ATGC, ATGA, ATGT, ATGG). These elements represent complete extended ASCII character set values.
b) The input information is then encrypted character-by-character using array generated in step 1. The basis is ASCII values of each character is matched with the element no. of the array of step 1.
   Encryption of the text “CSIR” in terms of DNA bases may be:
   TATGTTTCTATTTTAC where
   C is represented by DNA sequence TATG
   S is represented by DNA sequence TTTC
   I is represented by DNA sequence TATT
   R is represented by DNA sequence TTAC
c) If the information overflows the limits i.e. it cannot be synthesized in a single piece or because of any other problem, then the encrypted sequence is fragmented in a number segments.
d) Encrypted segment(s) is/are then flanked on each side with header and tail primers.
e) Synthesis of encrypted sequence(s) is then carried out using DNA synthesizer.
f) The synthesized DNA segment(s) is/are then be kept separately or can be mixed up with the enormous complex denatured DNA strands of genomic DNA of human or other organism. As the human genome contains about 3×10⁹ nucleotide pairs, fragmented & denatured human DNA provides a very complex background for storing encrypted DNA.
g) The encrypted DNA can then be transported on paper, cloths, buttons or through any other medium.

Isolation decryption of above encrypted DNA sequence

TATGTTTCTATTTTAC:

a) Isolation and amplification of encrypted DNA is done using known primers flanked at each end by PCR method.
b) Retrieved SM DNA is sequenced using DNA sequencer
c) Obtained sequence is interpreted (integrated if multi-segment before interpretation) using DNASTORE software. The basis for retrieval is a string of 4-bases each at a time is taken and matched with array as generated in step 1 of encryption and storage. The element number of matching value is taken and converted to its ASCII equivalent
   If the retrieved sequence is TATGTTTCTATTTTAC. The Decryption would be:
   first 4-bases i.e. “TATG” would be in the array storage and encryption 67=C
   next 4-bases i.e. “TTTC” would be in the array of storage and encryption 83=S
   next 4-bases i.e. “TATT” would be in the array storage and encryption 73=I
   next abases i.e. “TTAC” would be in the array of encryption 67=R
   Integration of above decrypted values in the same sequence as retrieved is “CSIR”.

### EXAMPLE 2

Some examples of DNA encryption for textual data

| Digital Information | Encrypted DNA sequence |
|---|---|
| WELCOME | TTAGTACATAGCTATGTACCTAACTACA |
| WORLD PEACE | TTAGTACCTTACTAGCTATAAGCTTTCCTAC |
| | ATAGGTATGTACA |
| INDIA | TATTTATCTATATATTTAGG |
| CSIR | TATGTTTCTATTTTAC |
| CSIO | TATGTTTCTATTTACC |

### EXAMPLE 3

A JPEG image encrypted in term of DNA bases

[image]

[image]

[image]

[image]

[image]

[image]

[image]

[image]

In example 2, a JPEG image if Indian Flag having file size of 1981 Bytes have been encrypted in terms of DNA bases. A total of 7924 DNA bases (4-base/Byte) are required to encrypt the complete image. Since the sequence is large, fragmenting the sequence into smaller segments is required.

### REFERENCES

1. Lalit M Bharadwaj*, Amol P Bhondekar, Awdbesh K. Shukla, Vijayender Bhalla and R P Bajpai. DNA-Based High-Density Memory Devices And Biomolecular Electronics At CSIO. Proc. SPIE: vol.493⁷, pp 319-325 (2002).
1. Clelland, C. T., Risea, V. & Bancroft, C. Hiding messages in DNA microdots. Nature. 399, 533-534(1999).
2. Bancroft, et al. DNA-based steganography, U.S. Pat. No. 6,312,911, November 2001.

1. A method for storing information in DNA using a unique sequence of 4-DNA bases for representing each character of extended ASCII character set comprising:
(a) producing a synthetic DNA molecule comprising encrypted digital information that can be decoded with the use of an encryption key, flanked on each side by a primer sequence; and
(b) storing the DNA molecule in a storage DNA, which consists of a mixture of homogenous/heterogeneous DNA

2. The method of claim 1 wherein the storage DNA is genomic DNA.

3. The method of claim 2 wherein the storage DNA is human DNA or any other organism's DNA.

4. The method of claim 1 wherein the storage DNA is synthetic.

5. The method of claim 1 wherein a software is provided to enable all 256 Extended ASCII characters to be defined in terms of DNA sequences.

6. The method of claim 1 wherein a minimum number of bases define each extended ASCII character.

7. The method of claim 1 wherein 4 sequences combinations result from one base A, T, G, C.

8. The method of claim 1 wherein with 2 bases 16 (4×4) different sequences are obtained.

9. The method of claim 1 wherein with three bases 64 (4×4×4) distinct sequences are obtained.

10. The method of claim 1 wherein with four bases 256 (4×4×4×4) distinct sequences are obtained.

11. The method of claim 1 wherein plain text/image or any digital information is encrypted in terms of DNA sequences using an encryption key software.

12. The method of claim 1 wherein the information is encrypted and fragmented in a number of segments if the information overflows the limits and cannot be synthesized in a single piece.

13. The method of claim 1 wherein synthesis of encrypted sequence(s) is carried out using DNA synthesizer.

14. The method of claim 1 wherein with a fixed number of different DNA primers sequence assigned a number, which resembles the segment position they represent.

15. The method of claim 1 wherein two tail primers are also provided, one of which resembles a continuation and other resembles termination segment.

16. The method of claim 1 wherein the DNA segment(s) is/are flanked by PCR primers at both ends with the header primers being attached at the beginning of segment and tail primers being attached at the end of the segment.

17. The method of claim 1 wherein SM DNA is mixed with complex denatured DNA strands of genomic DNA of human or other organism.

18. The method of claim 1 wherein a recipient knowing the sequences of both the primers [starting and tail] extracts the message, using PCR to isolate and amplify the encrypted DNA strand, followed by isolation and amplification of the DNA and sequencing using automated DNA sequencer, thereafter conversion of the DNA sequence obtained into digital message using encryption/decryption key.

19. A DNA molecule comprising an encrypted DNA sequence that can be decoded with the use of an encryption key, flanked on each side by polymerase chain reaction primer sequences wherein amplification of the DNA molecule and determination of the secret message DNA sequence and use of an encryption key, results in a decryption of the message.

20. A method as claimed in claim 1 where the method of encryption comprises:
a) encryption of a plain text/image or any digital information in terms of DNA sequences using encryption key, which first generates an array of 256 elements (unique 4-base per character), representing complete ended ASCII character set values;
b) encrypting of input information character-by-character using an array by matching the ASCII values of each character with the element number of the array;
c) fragmenting the encrypted sequence into a number of segments if the information overflows the limits and cannot be synthesized in a single DNA length;
d) flanking of the encrypted segment(s) on each side with header and tail primers;
e) synthesising of encrypted sequence(s) using DNA synthesizer;
f) mixing the synthesized DNA segment(s) with complex denatured DNA strands of genomic DNA of human or other organism,
g) transporting the encrypted DNA
h) Decrypting the encrypting DNA at the recipient end.

21. A method as claimed in claim 20 where the method of decryption comprises:
a) Isolation and amplification of encrypted DNA using known primers flanked at each end by PCR method;
b) sequencing of the retrieved encrypted DNA using DNA sequencer;
c) interpreting the obtained sequence after integration of multi-segment, if required using a predetermined encryption key;
